(19) United States
(12) Patent Application Publication
(10) Pub. No.: US 2026/0263414 A1
(43) Pub. Date: Sep. 10, 2026

(54) COMBINATIONS COMPRISING VITAMIN C AND LACTOBACILLUS RHAMNOSUS

(71) Applicant: DSM IP Assets B.V., Maastricht (NL)

(72) Inventors: Thanh-Van PHAM, Reinach (CH); Wilbert SYBESMA, Kaiseraugst (CH); Robert STEINERT, Kaiseraugst (CH); Ateequr REHMAN, Kaiseraugst (CH)

(21) Appl. No.: 18/871,658

(22) PCT Filed: Jun. 8, 2023
(86) PCT No.: PCT/EP2023/065397

§ 371 Date: Dec. 4, 2024

(30) Foreign Application Priority Data
Jun. 10, 2022 (EP) 22178527.2

(51) Int. Cl. A61K 31/375; A61K 35/747; A61P 1/00

(52) U.S. Cl. CPC A61K 31/375; A61K 35/747; A61P 1/00

(57) ABSTRACT

The present invention relates to a combination comprising vitamin C and Lactobacillus rhamnosus and its use for improving gut health in animals and humans. It was found that a combination of vitamin C and Lactobacillus rhamnosus, when delivered to the large intestine, increases the concentration of butyrate in the intestinal tract.

### FIELD OF THE INVENTION

The present invention relates to combinations comprising vitamin C and Lactobacillus rhamnosus, and their uses for improving gut health in animals and humans. It was found that a combination of vitamin C and Lactobacillus rhamnosus, when delivered to the large intestine, increases the concentration of butyrate in the intestinal tract.

### BACKGROUND OF THE INVENTION

Increasing evidence indicates that imbalances in the human gut microbiota (also referred to as “dysbiosis”) may be associated with Western diseases, including obesity and type 2 diabetes, as well as cardiovascular, autoimmune, and intestinal inflammatory disease. Thus, targeted modulation of the human gut microbiome intended to restore imbalances represents a potential therapeutic and preventive strategy and has attracted the attention of academics as well as those engaged in various industries. Public awareness and acceptance of substances that modulate the human gut microbiome continue to grow.

There is a consensus that certain live microorganisms have beneficial effects on human health via the production of metabolites such as butyrate. Butyrate, a short-chain fatty acid (SCFA), is an essential metabolite produced by the gut microbiome. Butyrate has many health benefits: butyrate is a source of energy for the cells of the gut lining, stimulates the production of glutathione, is a natural antioxidant, controls intestinal inflammation and supports a strong gut lining. It helps prevent cancer by preventing cancer cells from developing, and encourages the production of hormones for a healthy metabolism (Rinninella et al., What is the Healthy Gut Microbiota Composition?A Changing Ecosystem across Age, Environment, Diet, and Diseases (2019); Hamer H M et al., The role of butyrate on colonic function (2008)). Also, butyrate is effective against obesity and obesity-related diseases (Coppola S. et al., The Protective Role of Butyrate against Obesity and Obesity-Related Diseases (2021)).

The human gut microbiome produces hundreds of different metabolites, and it would be desirable to be able to selectively boost specific metabolites. In particular, it would be desirable to be able to increase the concentration of the SCFA butyrate in the intestine to enhance wellness, improve health, and support the immune system.

### SUMMARY OF THE INVENTION

The present invention relates to the following items:
1) Combination comprising vitamin C and Lactobacillus (Lacticaseibacillus) rhamnosus.
2) Combination according to item 1, wherein said combination comprises vitamin C and a Lactobacillus rhamnosus GG, preferably Lactobacillus rhamnosus DSM 32550.
3) Combination according to item 1 or item 2, wherein said combination is for simultaneous administration or delivery or consumption, and preferably wherein said combination is a fixed combination.
4) Combination according to item 1 or item 2, wherein said combination is for sequential administration or delivery or consumption, and preferably wherein said combination is a free combination.
5) Combination according to any one of item 1-4, wherein said combination is an oral dosage form, and wherein said combination is more preferably a solid oral dosage form.
6) Combination according to any one of items 1-5, wherein said combination is for administration or delivery to the large intestine.
7) Combination according to any one of item 1-6 for use as a medicament, a dietary supplement, or a food supplement.
8) Combination according to any one of item 1-7 for use in the treatment of a patient that is in need of increasing the concentration of butyrate in the large intestine.
9) Combination for the use according to item 8, wherein said patient is suffering from at least one or more of the following: irritable bowel syndrome, inflammatory bowel disease, colorectal cancer, graft-versus-host disease, diabetes, obesity, and neurodegenerative disorder.
10) Combination comprising vitamin C and Lactobacillus (Lacticaseibacillus) rhamnosus for use in increasing the concentration of butyrate in the large intestine of an animal, preferably a human, wherein said use comprises delivering or administering the vitamin C and the Lactobacillus rhamnosus to the large intestine.
11) Combination comprising vitamin C and Lactobacillus rhamnosus for the use according to item 10, wherein the vitamin C and the Lactobacillus rhamnosus are delivered or administered to the large intestine by a delayed-release formulation.
12) Combination comprising vitamin C and Lactobacillus rhamnosus for the use according to item 10 or item 11, wherein said use comprises delivering or administering the vitamin C and the Lactobacillus rhamnosus simultaneously and/or sequentially to the animal, preferably a human.
13) Combination comprising vitamin C and Lactobacillus rhamnosus for the use according to any one of item 10-12, wherein the animal, including a human, is experiencing a condition selected from: irritable bowel syndrome, inflammatory bowel disease, colorectal cancer, graft-versus-host disease, diabetes, obesity, and neurodegenerative disorder.
14) Combination comprising vitamin C and Lactobacillus rhamnosus for the use according to any one of item 10-13, wherein the Lactobacillus rhamnosus is a Lactobacillus rhamnosus GG, preferably Lactobacillus rhamnosus DSM 32550.

### BRIEF DESCRIPTION OF THE DRAWINGS

FIG. 1 Butyrate production (mM) (±stdev) during the 0-48 h time interval of the short-term colonic incubations for the different test conditions as compared to the respective control, averaged over the six different human donors (n=6). Average results have been included for Lactobacillus rhamnosus GG alone (L. rham) or when co-supplemented with vitamin C (L. rham+Vit C). Statistically significant differences between the test conditions and the control are indicated with ‘*’ (p<0.05).

### DETAILED DESCRIPTION OF THE INVENTION

Butyrate is a metabolite known for its beneficial effects on human health. The present inventors have found that vitamin C in combination with Lactobacillus rhamnosus can boost the concentration of butyrate in the large intestine, leading to an increase of butyrate levels in the gut.

Hence, in a first aspect, the present invention relates to combinations comprising vitamin C and Lactobacillus (Lacticaseibacillus) rhamnosus. Preferably, the Lactobacillus (Lacticaseibacillus) rhamnosus is a Lactobacillus rhamnosus GG strain; more preferably it is Lactobacillus rhamnosus DSM 32550. The combination is for simultaneous and/or sequential administration.

Patent claims relating to a “combination” are product claims. The product of the present invention comprises two active ingredients: a vitamin (vitamin C) and a probiotic (Lactobacillus rhamnosus). For simultaneous and/or sequential administration, see definitions and embodiments below.

Vitamin C, also known as L-ascorbic acid, is a water-soluble vitamin that is required for the biosynthesis of collagen, L-carnitine, and certain neurotransmitters. Vitamin C is also involved in protein metabolism. Further, vitamin C is an important physiological antioxidant. Vitamin C plays an important role in immune function and improves nutrient absorption. Vitamin C can be purchased from DSM GmbH. Alternative suppliers are, for example, TER Chemicals Distribution Group, BIOCHEM Bernburg GmbH, DVA International GmbH, Falken Trade GmbH, and Neupert Ingredients GmbH.

The most common Lactobacillus (Lacticaseibacillus) rhamnosus strain is Lactobacillus rhamnosus GG. It can be purchased, for example, from Chr. Hansen, Denmark, as LGG®. Lactobacillus (Lacticaseibacillus) rhamnosus DSM 32550 (Biocare Copenhagen, Denmark) has a genomic sequence which is 99.99% identical to the genomic sequence of LGG®. It can thus be considered that L. rhamnosus DSM 32550 is identical or equivalent to LGG® for practical purposes. Therefore, L. rhamnosus DSM 32550 will herein be referred to as a “Lactobacillus rhamnosus GG”.

Alternative Lactobacillus rhamnosus strains are, inter alia, Lactobacillus rhamnosus HN001 (Howaru; Danisco/DuPont), Lactobacillus rhamnosus GR-1® (Chr. Hansen, Denmark), and Lactobacillus rhamnosus Rosell-11 (Lallemand, Canada).

Lactobacillus (Lacticaseibacillus) rhamnosus DSM 32550 (Biocare Copenhagen) is a preferred strain according to the present invention. It has been deposited at Leibniz Institute DSMZ-German Collection of Microorganisms and Cell Cultures, Inhoffenstr. 7B, D-38124 Braunschweig, Germany, according to the Budapest Treaty on 6. July 2017. The accession number given by the International Depository Authority is DSM 32550.

In one embodiment, the combination of the present invention is for simultaneous administration. Preferably, the combination for simultaneous administration is a fixed combination. However, for simultaneous administration, a free combination can also be used.

In another embodiment, the combination is for sequential administration. The combination for sequential administration is a free combination.

Preferably, the combination is an oral dosage form; more preferably, it is a solid oral dosage form.

The combination of the present invention can be, for example, a pharmaceutical combination or composition, a dietary supplement, or a food supplement.

In another aspect, the present invention relates to vitamin C and Lactobacillus rhamnosus (i.e., a combination of vitamin C and Lactobacillus rhamnosus) for use as a medicament.

Preferably, the combinations of the invention (e.g. pharmaceutical combinations) are for use in the treatment of a patient that is in need of increasing the concentration of butyrate in the large intestine. In one embodiment, said patient is suffering from irritable bowel syndrome, inflammatory bowel disease, colorectal cancer, graft-versus-host disease, diabetes, obesity, or neurodegenerative disorders.

In a further aspect, the present invention relates to vitamin C and Lactobacillus rhamnosus (i.e., a combination of vitamin C and Lactobacillus rhamnosus) for use in improving gut health in an animal. Said improvement comprises or consists of increasing the concentration of butyrate in the large intestine of said animal. Specifically, the vitamin C and Lactobacillus rhamnosus is for use in increasing the concentration of butyrate in the large intestine (colon) of an animal, wherein said use preferably comprises delivering the vitamin C and Lactobacillus rhamnosus to the large intestine. Preferably, the animal is a human.

To achieve an increase of the concentration of butyrate in the large intestine, the vitamin C and Lactobacillus rhamnosus is preferably directly delivered to the large intestine. That is, the vitamin is delivered/administered in a manner such that the vitamin and the probiotic is not absorbed in the stomach and/or small intestine; rather the vitamin and the probiotic is delivered/administered to the distal intestinal tract, preferably the large intestine (colon). This is preferably done by delivering/administering the vitamin C and Lactobacillus rhamnosus in a delayed-release formulation. Oral administration is preferred.

In a preferred embodiment, the animal (including a human) is experiencing a condition selected from the group consisting of: irritable bowel syndrome, inflammatory bowel disease, colorectal cancer, graft-versus-host disease, diabetes, obesity, and neurodegenerative disorders.

Preferably, the Lactobacillus rhamnosus used is a Lactobacillus rhamnosus GG. Lactobacillus rhamnosus DSM 32550 is particularly preferred.

In another aspect, the present invention relates to a method of increasing the concentration of butyrate in the intestine, preferably the large intestine, comprising administering to the animal an effective dose of vitamin C and Lactobacillus rhamnosus (preferably, Lactobacillus rhamnosus GG; in particular Lactobacillus rhamnosus DSM 32550). The method is for improving intestinal health in an animal, including a human, wherein said improvement comprises increasing the concentration of butyrate in the large intestine. Preferably, the animal is a human. Preferably, the vitamin C and the Lactobacillus rhamnosus is delivered directly to the large intestine. Delivery to the large intestine can be achieved by administering the vitamin C and the Lactobacillus rhamnosus as a delayed-release formulation.

The methods of the invention can be used to treat, prevent, and/or lessen the symptoms of irritable bowel syndrome, inflammatory bowel disease, colorectal cancer, graft-versus-host disease, diabetes, obesity, and neurodegenerative disorders in an animal, including a human, in need thereof.

In a further aspect, the present invention relates to the use of vitamin C and Lactobacillus rhamnosus for increasing the concentration of butyrate in the large intestine of an animal, preferably a human, wherein said use comprises delivering the vitamin C and the Lactobacillus rhamnosus to the large intestine. Preferably, the use comprises delivering the vitamin C and Lactobacillus rhamnosus to the large intestine by a delayed-release formulation. Preferably, the animal, including a human, is experiencing at least one condition selected from the group consisting of: irritable bowel syndrome, inflammatory bowel disease, colorectal cancer, graft-versus-host disease, diabetes, obesity, and neurodegenerative disorders.

In the combinations, uses, and methods of the invention, preferably, the vitamin C (ascorbic acid) dose is up to 2000 mg/day, preferably 100-2000 mg/day; more preferably 200-1000 mg/day. In one embodiment, vitamin C is dosed/administered in an amount such that its local concentration in the colon is at least 0.05 g/L, preferably at least 0.1 g/L, most preferably at least 0.33 g/L. Preferred local concentrations in the colon range from about 0.05 g/L to about 1.5 g/L, more preferably from about 0.1 g/L to about 1 g/L, most preferably from about 0.2 g/L to about 0.5 g/L.

The dosage of the Lactobacillus rhamnosus can be up to 5E+10 cfu/day. Preferably, the dosage range is from 1E+08 to 1E+10 cfu/day, more preferably from 1E+09 to 5E+10 cfu/day. Preferably, the Lactobacillus rhamnosus is a Lactobacillus rhamnosus GG. Lactobacillus rhamnosus DSM 32550 is particularly preferred.

### DEFINITIONS AND EMBODIMENTS

As used throughout, the following definitions apply: Patent claims relating to a “combination” or “pharmaceutical combination” are product claims. The product of the present invention comprises two active ingredients: a vitamin (vitamin C) and a probiotic (Lactobacillus rhamnosus).

A “combination for simultaneous administration” or a “combination for simultaneous consumption” is a combination that is suitable for simultaneous administration or consumption, respectively. By “simultaneous administration” or “simultaneous consumption”, it is meant that the vitamin and the probiotic bacteria are administered/consumed on the same day (i.e., with 24 hours). Said two active ingredients can be administered/consumed at the same time (for fixed combinations) or one at a time (for free combinations). For example, the vitamin can be administered/consumed in one pill or tablet, while the probiotic is administered/consumed in another pill or tablet, wherein both pills/tablets are administered/consumed within 24 hours. In another example, the vitamin and the probiotic are formulated in the same composition and are administered/consumed at exactly the same time.

A “combination for sequential administration or consumption” is a combination that is suitable for sequential administration or consumption, respectively. By “sequential administration” or “sequential consumption”, it is meant that during a period of two or more days of continuous treatment, only one of the vitamin and the probiotic is administered/consumed on any given day.

By way of example, the vitamin can be administered/consumed on day one, and the probiotic is administered/consumed only the next day (i.e., after more than 24 hours), or even later. The active ingredients can be administered/consumed in any order.

A “fixed combination” is a combination that delivers both actives (i.e., the vitamin and the probiotic) at the same time to a patient. A solid oral dosage form (e.g., a tablet or capsule) comprising both, the vitamin and the probiotic, is an example of a fixed combination. A liquid oral dosage form (e.g., oral drops) comprising both, the vitamin and the probiotic, is another example of a fixed combination.

A “free combination” is a combination that allows to administer/consume both actives (i.e., the vitamin and the probiotic) separately, i.e. one at a time. Treatment regimens in which the vitamin and the probiotic are not administered/consumed by the same route and/or are not administered/consumed at the same time require free combinations.

Simultaneous administration/consumption can be done both by using a fixed combination and a free combination. Sequential administration/consumption requires a free combination; fixed combinations are not suitable for sequential administration/consumption. Hence, free combinations are more versatile: they are suitable for sequential administration/consumption and—if both actives are administered/consumed on the same day—also for simultaneous administration/consumption. Fixed combinations are only suitable for simultaneous administration/consumption if both ingredients (i.e., the vitamin and the probiotic) are to be administered/consumed at the same time of the same day; if, however, the vitamin and the probiotic are to be administered/consumed on the same day but separately, fixed combinations are not suitable.

By “separate administration/consumption”, it is meant that the vitamin and the probiotic are administered one at a time. Thus, separate administration/consumption can refer to both sequential administration/consumption and—when referring to the administration/consumption of both actives on the same day but one at a time—also to simultaneous administration/consumption.

“Administering” or “administration” means to give or to deliver an active to a human or animal; likewise, the human or animal can take (consume) the active.

The term “vitamin C” which can be used interchangeably with “ascorbic acid” also includes pharmaceutically acceptable salts thereof (e.g., sodium ascorbate and calcium ascorbate) and pharmaceutically acceptable esters thereof (in particular ascorbyl palmitate) and other pharmaceutically acceptable forms.

“SCFAs” (short for “short chain fatty acids”) are fatty acids with fewer than six carbon atoms. They are derived from intestinal microbial fermentation of indigestible foods. The three most common SCFAs are acetate, propionate, and butyrate. Butyrate, also known under the name butyric acid, or butanoic acid, is a SCFA that serves as a primary energy source for colonocytes and also maintains intestinal homeostasis through anti-inflammatory actions.

To “increase the concentration of butyrate” means to increase the level (or amount) of butyrate compared to the respective control (i.e., the level/amount of butyrate when the combination of vitamin C and Lactobacillus rhamnosus has not been added).

The term “intestine” (or “gut”) as used herein refers to the portion of the gastrointestinal tract consisting of the small intestine and the large intestine. The “large intestine” (intestinum crassum) is the lower part of the gastrointestinal tract and is also referred to herein as “colon”.

“Direct delivery” or “directly delivered” means that the vitamin is formulated in a manner such that the vitamin is not absorbed in the stomach and/or small intestine; rather the vitamin is made available in the distal intestinal tract, preferably the large intestine (colon), where it is available to the microbiome. The vitamin is not part of a person's usual daily nutritional requirements (generally obtained through diet and conventional vitamin supplementation), and is administered in excess thereof. For human use, the preferred method according to the present invention is through a form which delays release until the large intestinal tract (colon) is reached. Alternatively, a large enough dose can be administered, so that only a portion of the administered vitamin is absorbed in the proximal small intestine, and the remainder, which is an effective dose, is available to the large intestinal tract; although not preferred, the latter method of delivery can be used for humans as well. With respect to the probiotic, “direct delivery” or “directly delivered” means that the probiotic is formulated in a manner such that it is not released in the stomach and/or small intestine but rather it is made available in the distal intestinal tract, preferably the large intestine (colon).

A used herein, “delayed release” refers to the release of the vitamin and/or the probiotic at a time later than immediately after administration. Preferably, “delayed release” means delivery of the vitamin (and/or probiotic), upon oral administration, to the large intestine (colon) in a delayed manner relative to an immediate release formulation.

An “enteric layer” or “enteric coating” is a layer surrounding a core, wherein the core comprises the active agent and the layer confers resistance to gastric juice.

“Prevent” can include lessening the risk of an adverse condition occurring, lessening the symptoms of an adverse condition, lessening the severity of an adverse condition, and prolonging the time for occurrence of an adverse condition.

“Oral formulation” means that the vitamin and/or probiotic is formulated for oral administration/consumption.

“Co-administering” or “co-administration” means that the vitamin and/or the probiotic is delivered/administered/consumed simultaneously (i.e., together), or separately but within a time frame of 24 hours. The vitamin can be delivered/administered/consumed first. Likewise, the probiotic can be delivered/administered/consumed first.

“Lactobacillus rhamnosus” was recently renamed “Lacticaseibacillus rhamnosus”; both names are used interchangeably herein, and both can be abbreviated as “L. rhamnosus”.

### Doses

Preferably, vitamin C is administered in an amount such that its local concentration in the colon is at least 0.05 g/L, preferably at least 0.1 g/L, most preferably at least 0.33 g/L. Preferred local concentrations in the colon range from about 0.05 g/L to about 1.5 g/L, more preferably from about 0.1 g/L to about 1 g/L, most preferably from about 0.2 g/L to about 0.5 g/L. Specific dosages per day can range up to 2000 mg/day, preferably 100-2000 mg/day; more preferably 200-1000 mg/day.

The dosage of the probiotic can be up to 5E+10 cfu/day. Preferably, the dosage range of the probiotic is from 1E+08 to 1E+10 cfu/day, more preferably from 1E+09 to 5E+10 cfu/day.

### Formulations

The vitamin (vitamin C) and/or the probiotic (Lactobacillus rhamnosus), preferably both, is (are) preferably present in a formulation which allows the vitamin (and/or probiotic) to be available predominantly in the large intestine.

Oral formulations are preferred. Other formulations include non-oral routes, such as via suppositories or injections.

For human use, the preferred method is through a delayed-release form which delays delivery until the intestinal tract is reached. For non-human animals, a preferred delivery includes a method of administering a large enough dose so that only a portion of the vitamin and/or probiotic delivered is absorbed in the stomach, and the remainder, which is an effective dose, is available to the intestinal tract; although not preferred, this method of delivery can be used for humans as well.

Delayed-release formulations are known in the art. Preferably, the delayed-release formulations have an enteric coating (also referred to as enteric layer).

In one embodiment of the present invention, the vitamin and/or probiotic, preferably both, is in a formulation comprising an enteric capsule, filled with a composition comprising the vitamin and/or probiotic. The enteric capsule confers resistance against the acidic environment of the stomach.

For example, soft gel formulations may deliver the active agent in solution and yet offer advantages of solid dosage forms.

In another embodiment, the formulation is a tablet comprising (i) a core comprising the vitamin and/or the probiotic, and (ii) a delayed-release coating such as an enteric coating. This may be a hard gel capsule.

Alternatively, for direct colon delivery, a matrix-based delivery system can be used. Matrix based systems have no discrete layer of coating material, but the active agent (i.e., the vitamin and/or the probiotic) is more or less homogenously distributed within the matrix. Further, there are colon-release systems that embed the active agent in e.g. in a fiber matrix (enzyme-triggered) and an enteric coating on top.

The release of the vitamin and/or probiotic may be delayed until the small intestine. In another embodiment, the release is delayed until the distal small intestine. In yet another, preferred embodiment, the release of the vitamin and/or probiotic is delayed until the colon (large intestine).

In a preferred embodiment for humans, the vitamin and/or probiotic is formulated in a solid dosage form for oral administration. The formulation may be in the form of a capsule, pellet, bead, sphere, mini spheres, tablet, mini tablet, or granule, optionally coated with a delayed release coating that prevents the release of the active agent before the small intestine, preferably before the colon.

Coating, or matrix materials for the delayed release of the vitamin and/or probiotic, in particular for targeted release in the ileum or the large intestine upon oral administration are known in the art. They can be subdivided into coating materials that disintegrate above a specific pH, coating materials that disintegrate after a specific residence time in the gastrointestinal tract and coating materials that disintegrate due enzymatic triggers specific to the microflora of a specific region of the intestines. Coating materials from different categories are commonly used in combinations.

Coating materials of the different categories for targeting to the large intestine have been reviewed for example in Bansal et al. (Polim. Med. 2014, 44, 2, 109-118). In one embodiment of the present invention, the delayed-release coating comprises at least one component selected from coating materials that disintegrate pH-dependently, coating materials that disintegrate time-dependently, coating materials that disintegrate due to enzymatic triggers in the intestinal environment (e.g., in the intestinal environment of the ileum and the large intestine), and combinations thereof.

Coating materials that disintegrate pH-dependently include polyvinyl acetate phthalate, cellulose acetate trimellitate, hydroxypropyl methylcellulose phthalate HP-50, HP-55 or HP-55S, cellulose acetate phthalate, shellac, hydroxypropyl methylcellulose acetate succinate (HPMCAS), poly(methacrylic acid, ethyl acrylate) 1:1 (Eudragit® L100-55, Eudragit® L30D-55), poly(methacrylic acid, methyl methacrylate) 1:1 (Eudragit® L-100, Eudragit® L12.5), poly(methacrylic acid, methyl methacrylate) 1:2 (Eudragit® S-100, Eudragit® S12,5, and Eudragit® FS30D). Coating materials that disintegrate time-dependently include Eudragit® RL, Eudragit® RS, and ethylcellulose. Coating materials that disintegrate due to enzymatic triggers in the large intestinal environment include chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, and azo compounds being degraded by azo bonds splitting bacteria.

The following non-limiting examples are presented to better illustrate the invention.

### EXAMPLES

The aim of this study was to investigate the effect of a combination of vitamin C and Lactobacillus rhamnosus on the metabolic activity of the gut microbiota in a short-term batch fermentation experiment.

### Materials and Methods

### Design of the Batch-Fermentation Experiment (Colon Model)

A short-term batch-fermentation experiment was carried out by ProDigest, consisting of a colonic incubation of a representative dose of a selected vitamin with a representative bacterial inoculum under simulated conditions for the proximal large intestine. In the current experiment, the bacterial inocula were derived from fresh fecal samples of six different healthy adult donors. Incubations were performed as described previously (Van den Abbeele, P.; Taminiau, B.; Pinheiro, I.; Duysburgh, C.; Jacobs, H.; Pijls, L.; Marzorati, M. Arabinoxylo-Oligosaccharides and Inulin Impact Inter-Individual Variation on Microbial Metabolism and Composition, Which Immunomodulates Human Cells. J. Agric. Food Chem. 2018, 66, 1121-1130). At the start of the short-term colonic incubation, fresh fecal material from six healthy human donors was collected and upon preparation of an anaerobic fecal slurry, this slurry was inoculated at 10 vol % in a SHIME nutritional medium containing basal nutrients containing 3.5 g/L K2HPO4, 10.9 g/L KH2PO4, 2 g/L NaHCO₃(Chem-lab NV, Zedelgem, Belgium), 2 g/L yeast extract, 2 g/L peptone (Oxoid, Aalst, Belgium), 1 g/L mucin (Carl Roth, Karlsruhe, Germany), 0.5 g/L L-cysteine and 2 mL/L Tween80 (Sigma-Aldrich, Bornem, Belgium). All test ingredients (i.e., probiotic strain, vitamin C) were also added to the SHIME medium. Furthermore, M-SHIME® technology was incorporated in the current experiment by adding mucin-covered microcosms (modeling the mucus of the colon) to the incubations as described previously (Van den Abbeele, P., et al. (2013). Butyrate-producing Clostridium cluster XIVa species specifically colonize mucins in an in vitro gut model. The ISME Journal 7(5), 949-961). Incubations were performed for 48 h, at 37° C., under shaking (90 rpm) and anaerobic conditions.

In this study, the Lactobacillus rhamnosus GG equivalent Lactobacillus rhamnosus DSM 32550 (Biocare Copenhagen) was added to the colon model containing the donor samples, alone or in combination with vitamin C (see Table 1), and the level of butyrate was analyzed. Experiments were performed in single repetition.

Lactobacillus rhamnosus DSM 32550 has a genomic sequence which is 99.99% identical to the genomic sequence of LGG®. It can therefore be considered that L. rhamnosus DSM 32550 is identical or equivalent to LGG® for practical purposes. In the Examples herein, Lactobacillus rhamnosus DSM 32550 will thus be referred to as a Lactobacillus rhamnosus GG strain.

TABLE 1
Experiment layout

| | Donor | Donor | Donor | Donor | Donor | Donor |
|---|---|---|---|---|---|---|
| Test ingredient | 1 | 2 | 3 | 4 | 5 | 6 |
| Lactobacillus rhamnosus | x | x | x | x | x | x |
| GG only (Control) | | | | | | |
| Lactobacillus rhamnosus | x | x | x | x | x | x |
| GG + Vitamin C | | | | | | |

The examined probiotic strain Lactobacillus rhamnosus DSM 32550 was added to the SHIME medium as an overnight grown culture at a concentration of 1*109 CFU. The probiotic strain was either supplemented alone or in combination with vitamin C. Vitamin C (Ascorbic acid, DSM) was added at a concentration of 0.333 g/L which translates into a 200 mg dose, given that the colon volume is about 600 ml.

### Analysis of Butyrate Level

Samples for butyrate analysis were analyzed after Oh and 48 h of incubation. Butyrate was extracted from the samples with diethyl ether, after the addition of 2-methyl hexanoic acid as an internal standard. Extracts were analysed using a GC-2014 gas chromatograph (Shimadzu, 's-Hertogenbosch, the Netherlands), equipped with a capillary fatty acid-free EC-1000Econo-Cap column (dimensions: 25 mm×0.53 mm, film thickness 1.2 mM; Alltech, Laarne, Belgium), a flame ionization detector, and a split injector. The injection volume was 1 mL and the temperature profile was set from 110 to 160° C., with a temperature increase of 6° C. min-1. The carrier gas was nitrogen, and the temperature of the injector and detector were 100 and 220° C., respectively.

### Statistics

Statistical analysis was performed to investigate the average effect of the test products. For this purpose, the average over the six donors was calculated for each endpoint. Paired t-tests were conducted to evaluate the potential effect of the test products as compared to the control as well as to compare the different test products with each other. Differences were considered statistically significant if the p-value was less than 0.05.

### Results

Supplementation of a Combination of Lactobacillus rhamnosus and Vitamin C Increased the Concentration of Butyrate

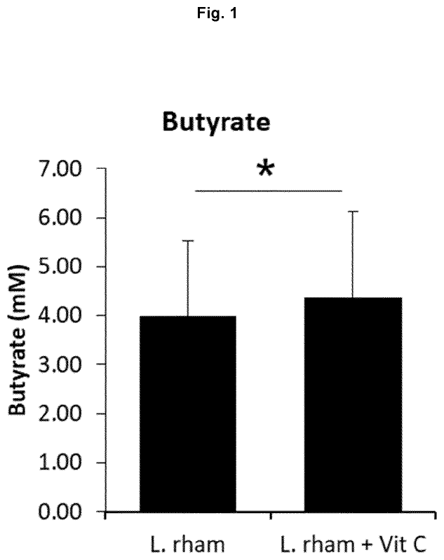

As can be taken from FIG. 1, supplementation of a combination of vitamin C and Lactobacillus rhamnosus GG led to a significantly increased butyrate concentration as compared to the respective control (Lactobacillus rhamnosus GG alone).

1. Combination comprising:
i) vitamin C, and
ii) Lactobacillus rhamnosus.

2. Combination according to claim 1, wherein said combination comprises:
i) vitamin C, and
ii) Lactobacillus rhamnosus GG.

3. Combination according to claim 1, wherein said combination is for simultaneous administration.

4. Combination according to claim 1, wherein said combination is for sequential administration.

5. Combination according to claim 1, wherein said combination is an oral dosage form, and wherein said combination is more preferably a solid oral dosage form.

6. Combination according to claim 1, wherein said combination is for administration to the large intestine.

7. Combination according to claim 1 for use as a medicament, a dietary supplement, or a food supplement.

8. Combination according to claim 1 for use in the treatment of a patient that is in need of increasing the concentration of butyrate in the large intestine.

9. Combination for the use according to claim 8, wherein said patient is suffering from irritable bowel syndrome, inflammatory bowel disease, colorectal cancer, graft-versus-host disease, diabetes, obesity, or neurodegenerative disorders.

10. Combination comprising vitamin C and Lactobacillus rhamnosus for use in increasing the concentration of butyrate in the large intestine of an animal, preferably a human, wherein said use comprises administering or delivering the vitamin C and the Lactobacillus rhamnosus to the large intestine.

11. Combination comprising vitamin C and Lactobacillus rhamnosus for the use according to claim 10, wherein the vitamin C and the Lactobacillus rhamnosus are administered or delivered to the large intestine by a delayed-release formulation.

12. Combination comprising vitamin C and Lactobacillus rhamnosus for the use according to claim 10, wherein said use comprises administering or delivering the vitamin C and the Lactobacillus rhamnosus simultaneously and/or sequentially to the animal, preferably a human.

13. Combination comprising vitamin C and Lactobacillus rhamnosus for the use according to claim 10, wherein the animal, including a human, is experiencing at least one condition selected from: irritable bowel syndrome, inflammatory bowel disease, colorectal cancer, graft-versus-host disease, diabetes, obesity, and neurodegenerative disorders.

14. Combination comprising vitamin C and Lactobacillus rhamnosus for the use according to claim 10, wherein the Lactobacillus rhamnosus is Lactobacillus rhamnosus GG.
